SEQUENCE LISTING (1 sequence)

SEQ ID NO: 1 - DNA, 615 bp, synthetic construct
  source 1..615: mol_type = other DNA
atgaaattctcttccgctttggttctatctgctgttgccgctactgctcttgctgagagtatcaccaccaccatcactgccaccaagaacggtcatgtctacactaagactgtcacccaagatgctacttttgtttggggtggtgaagactcttacgccagcagcacttctgccgctgaatcttctgccgccgaaacttctgccgccgaaacctctgctgccgctaccacttctgctgccgctaccacttctgctgctgagacttcttctgctgctgagacttcttctgctgatgaaggttctggttctagtatcactaccactatcactgccaccaagaacggtcacgtctacactaagactgtcacccaagatgctacttttgtctggactggtgaaggcagcagcaacacctggtctccaagtagtacttctaccagctcagaagctgctacctcttctgcttcaaccactgcaaccaccactgctgaaacttcttcctctgctacatcctctagcaccgctgaattatctagttacaccggtgctgctgatgccatcaccgctggtactggtttgatgggtgctgcccttgctgccgtcatgttattgtaa

(19) United States
(12) Patent Application Publication
(10) Pub. No.: US 2025/0115921 A1
(43) Pub. Date: Apr. 10, 2025

(54) YEAST GENE ScFIT3 FOR REGULATING AND CONTROLLING CADMIUM RESISTANCE OF ORGANISM AND APPLICATION THEREOF

(71) Applicants: Shandong Huaneng Power Generation Co.,Ltd., Jinan (CN); Huaneng Zhanhua Photovoltaic Power Generation Co., Ltd, Binzhou (CN); Shandong Agricultural Machinery Science and Research Institute, Jinan (CN); Shandong Academy of Agricultural Sciences, Jinan (CN)

(72) Inventors: Jianwei GAO, Jinan (CN); Lilong HE, Jinan (CN); Ali ANWAR, Jinan (CN); Jingjuan LI, Jinan (CN); Yihui ZHANG, Jinan (CN); Cheng LI, Jinan (CN); Fengde WANG, Jinan (CN); Shu ZHANG, Jinan (CN); Jihua AN, Jinan (CN); Xianxin MA, Jinan (CN); Yuezhong PEI, Jinan (CN); Jiwei LIU, Jinan (CN)

(73) Assignees: Shandong Huaneng Power Generation Co.,Ltd., Jinan (CN); Huaneng Zhanhua Photovoltaic Power Generation Co., Ltd, Binzhou (CN); Shandong Agricultural Machinery Science and Research Institute, Jinan (CN); Shandong Academy of Agricultural Sciences, Jinan (CN)

(21) Appl. No.: 18/983,676

(22) Filed: Dec. 17, 2024

Related U.S. Application Data
(63) Continuation of application No. PCT/CN2023/125445, filed on Oct. 19, 2023.

(30) Foreign Application Priority Data
Nov. 24, 2022 (CN) 202211483455.4

(51) Int. Cl. C12N 15/81; C12P 19/34

(52) U.S. Cl. CPC C12N 15/81; C12P 19/34

(57) ABSTRACT

The present invention relates to a yeast gene ScFIT3 for regulating and controlling cadmium resistance of an organism and an application thereof. The yeast gene ScFIT3 for regulating and controlling the cadmium resistance of the organism has a nucleotide sequence as shown in SEQ ID NO.1. The present invention discloses, for the first time, the application of the yeast gene ScFIT3 in enhancing cadmium resistance of the organism. The yeast gene ScFIT3 is linked to an expression vector and transformed into a budding yeast, thereby completing construction of a cadmium-resistant yeast. The growth of the budding yeast transformed with the yeast gene ScFIT3 is significantly superior to that of a wild-type budding yeast, indicating that the budding yeast transformed with the yeast gene ScFIT3 has good resistance to cadmium stress, significantly enhances a capability of resisting heavy metal cadmium, and reduces accumulation and absorption of cadmium.

### CROSS-REFERENCE TO RELATED APPLICATIONS

The application claims priority to Chinese patent application No. 202211483455.4, filed on Nov. 24, 2022, the entire contents of which are incorporated herein by reference.

### SEQUENCE LISTING

The sequence listing xml file submitted herewith, named “SEQUENCE_LISTING.xml”, created on Nov. 29, 2024, and having a file size of 2,461 bytes, is incorporated by reference herein.

### TECHNICAL FIELD

The present invention relates to a yeast gene ScFIT3 for regulating and controlling cadmium resistance of an organism and an application thereof, belonging to the field of plant genetic engineering.

### BACKGROUND

In recent years, due to the irrational application of agricultural chemicals such as pesticides and fertilizers, the amount of heavy metal pollutants accumulating in soil of arable land and facility agricultural land has been increasing year by year, posing a serious threat to the lives and physical health of people. Heavy metal pollution is concealed, long-term, and irreversible, and its severity has not yet attracted high attention as compared to pesticide residues. Cadmium, listed as a class I carcinogen by the World Health Organization, is the major element causing heavy metal pollution of the soil, with a standard-exceeding rate of 7.0% at points nationwide, which is much higher than that of other measured inorganic pollutants (Bulletin of National Soil Pollution Status Survey, 2014).

At present, the use of molecular biology to improve plants is an effective means to deal with cadmium pollution in the plants. In recent decades, botanists and crop scientists have screened many cadmium transporter proteins, such as ABC protein family, HMA protein family, NRAMP protein family, etc., from different plants and applied them to plant and crop improvement processes. However, most of these proteins transport cadmium ions to vacuoles of the plants or to certain parts of the plants, merely isolating the cadmium ions, which poses a risk of cadmium pollution to the food chain. Yeast, as the simplest eukaryote and a common ancestor of other eukaryotes, contains a large number of unknown functional genes waiting to be explored. Further research is urgently needed to discover new genes that affect cadmium stress and cadmium accumulation of the plants.

### SUMMARY

In view of the deficiencies of the prior art, the present invention provides a yeast gene ScFIT3 for regulating and controlling cadmium resistance of an organism and an application thereof.

### The Present Invention Adopts the Following Technical Solution

A yeast gene ScFIT3 for regulating and controlling cadmium resistance of an organism and an application thereof, where the yeast gene ScFIT3 for regulating and controlling the cadmium resistance of the organism has a nucleotide sequence as shown in SEQ ID NO.1.

An expression vector containing the yeast gene ScFIT3 for regulating and controlling cadmium resistance of a plant.

According to the present invention, preferably, a method for constructing the expression vector includes the following steps:
(1) extracting an RNA from a budding yeast, and then performing reverse transcription on the RNA to obtain a cDNA;
(2) performing PCR amplification by using the cDNA as a template to obtain a sequence of a yeast gene ScFIT3, where PCR primer sequences are as follows:

Forward primer:
5′-ATGAAATTCTCTTCCGCTTT-3′,
Reverse primer:
5′-TTACAATAACATGACGGCAG-3′;

(3) linking the yeast gene ScFIT3 to a pRS416 plasmid to obtain the expression vector containing the yeast gene ScFIT3.

An application of the yeast gene ScFIT3 described above in enhancing cadmium resistance of an organism.

According to the present invention, preferably, a route of the application includes: transforming the expression vector containing the yeast gene ScFIT3 into a host organism.

According to the present invention, preferably, the host organism includes a plant and a microorganism.

Further preferably, the microorganism is a yeast.

### Beneficial Effects

The present invention discloses, for the first time, an application of the yeast gene ScFIT3 in enhancing cadmium resistance of the organism. The yeast gene ScFIT3 is linked to an expression vector and transformed into a budding yeast, thereby completing construction of a cadmium-resistant yeast. The growth of the budding yeast transformed with the yeast gene ScFIT3 is significantly superior to that of a wild-type budding yeast, indicating that the budding yeast transformed with the yeast gene ScFIT3 has good resistance to cadmium stress, significantly enhances a capability of resisting heavy metal cadmium, and reduces accumulation and absorption of cadmium.

### BRIEF DESCRIPTION OF THE DRAWINGS

FIG. 1 shows a graph of growth results of a yeast transformed with a gene ScFIT3 and a JRY472 yeast growing on an SC solid screening medium containing CdCl₂ (50 μM) and a normal SC solid screening medium;

in the figure, triangular symbols represent a ten-fold decrease in concentration (an initial concentration being 0.3 OD600).

FIG. 2 shows a Cd content in a budding yeast JRY472 transformed with a yeast gene ScFIT3 after 14 h of growth under the same cadmium treatment conditions. Error bars represent ±SD statistics of three independent repeated tests. P values of T-test: comparison of cadmium contents of a transgenic yeast and a wild-type yeast, **P<0.01.

### DETAILED DESCRIPTION OF THE EMBODIMENTS

The present invention is further described below with reference to the embodiments. However, the scope of the present invention is not limited to the following embodiments. Those skilled in the art can understand that various changes and modifications can be made to the present invention without departing from the spirit and scope of the present invention. A general and/or specific description of materials and testing methods used in tests is provided by the present invention. Although many materials and operating methods used to achieve the purposes of the present invention are well known in the art, the present invention is still described herein in as much detail as possible.

The budding yeast JRY472 described in the embodiments has been disclosed in the reference A Mitochondrial Pyruvate Carrier Required for Pyruvate Uptake in Yeast, Drosophila, and Humans, 2012.

### Embodiment 1

1. A method of extracting an RNA from a budding yeast JRY472 specifically included the following steps:
weighing 5 g of budding yeast JRY472 cell powder, suspending the powder in 30 mL of a 0.04M NaOH solution, performing uniform grinding in a mortar to obtain suspension, transferring the suspension to a conical flask, heating in a boiling water bath for 30 min, cooling, transferring to a centrifuge tube, performing centrifugation at 3000 r/min for 15 min, and then slowly pouring supernatant into 10 mL of acidic ethanol, stirring while adding; after addition of the supernatant is complete, standing, performing centrifugation at 3000 r/min for 3 min after the RNA is precipitated, removing the supernatant, washing a precipitate twice with 95% ethanol, washing the precipitate once with ethyl/ether, and then transferring the precipitate to a Buchner funnel with ethyl/ether for suction filtration, drying the precipitate in air, dissolving a total RNA sample in 50 μL of DEPC-treated ddH₂O, and storing a prepared total RNA sample in an ultra-low temperature freezer at −80° C. for future use.
2. Reverse transcription PCR

Reverse transcription PCR amplification (a two-step method) was performed by using the total RNA sample as a template, and the specific reverse transcription PCR amplification system and conditions were as follows:

Reaction steps were as follows:
Step1:

| Reagent | Usage amount | |
|---|---|---|
| 5 × gDNA Eraser Buffer | 2 | μL |
| gDNA | 1 | μL |
| Total RNA | 1 | μg |
| RNase Free ddH₂O | 7 | μL |
| Total | 10 | μL |

Reaction conditions: warm bath at 42° C. for 2 min, storage at 4° C.
Step2:

| Reagent | Usage amount | |
|---|---|---|
| Reaction solution from Step 1 | 10 | μL |
| 5 × PrimeScript Buffer 2 | 4 | μL |
| PrimeScript RT Emzyme Mix | 1 | μL |
| RT Primer Mix | 1 | μL |
| RNase Free ddH₂O | 4 | μL |
| Total | 20 | μL |

Reaction conditions: warm bath at 37° C. for 15 min, reaction for 5 s at 85° C.

A total cDNA of the budding yeast JRY472 was obtained by the above methods, and the reagents in Step1 and Step2 were available from Prime Script™ II 1st Strand cDNA Synthesis Kit of the TAKALA company.

### Embodiment 2

1. A method for constructing an expression vector containing a yeast gene ScFIT3 for regulating and controlling cadmium resistance of a plant specifically included the following steps:
(1) performing PCR amplification by using a yeast cDNA as a template to obtain a sequence (SEQ ID NO.1) of a yeast gene ScFIT3, where primers for the PCR amplification were as follows:

Forward primer:
5′-aatctagaactagtgATGAAATTCTCTTCCGCTTT-3′,
Reverse primer:
5′-gcagcccgggggatcTTCCTCCTCCTCCTCCTCCTTACAATAA-
CATGACGGCAG-3′;

The PCR system was as follows:

| Reagent | Usage amount | |
|---|---|---|
| Phusion DNA Polymerase | 0.5 | μL |
| 5 × phusion GC buffer | 10 | μL |
| dNTP mix (2.5 mM) | 4 | μL |
| Forward primer (10 μM) | 2 | μL |
| Reverse primer (10 μM) | 2 | μL |
| DMSO (optional for GC buffer) | 1.5 | μL |
| Template DNA (100 ng) | 2 | μL |
| ddH₂O | 28 | μL |
| Total | 50 | μL |

The PCR conditions were as follows: predegeneration, 98° C., 30 s; degeneration, 98° C., 10 s; annealing, 55° C. to 65° C., 30 s; extension, 72° C., 1 min (35 cycles); termination of extension, 72° C., 5 min to 10 min; finally, heat preservation at 4° C.
(2) performing enzyme digestion on a pRS416 plasmid (containing a pGPD promoter) by using a restriction endonuclease BamH1, and linking the sequence of the yeast gene ScFIT3 to the pRS416 plasmid by using a homologous recombinase (In-Fusion) to obtain the expression vector containing the yeast gene ScFIT3.

The linking system was as follows:

| Reagent | Usage amount | |
|---|---|---|
| 5 × In-Fusion HD enzyme premix | 2 | μL |
| pRS416 plasmid | 1 | μL |
| BrMYB116 gene fragment | 7 | μL |
| Total | 10 | μL |

Reaction conditions for linking: 50° C., 15 min; storage at 4° C.
2. Preparation of transgenic yeasts

The preparation specifically included: dipping a stock solution of a budding yeast JRY472 stored at −80° C. to streak on a YPDA solid medium, and inverting and culturing the stock solution at 30° C. for 4 days; selecting a single colony of the budding yeast JRY472, placing same into 5 mL of a YPDA liquid medium, and performing shake culture at 30° C. and 250 rpm for 10 h; taking 3 mL of a bacteria solution of the budding yeast JRY472, placing the bacteria solution into 50 mL of the YPDA liquid medium for expanded culture, and performing shake culture at 30° C. and 250 rpm until OD600≤0.5 is achieved; and then, performing centrifugation on the bacteria solution of the budding yeast JRY472 at 25° C. and 3000 rpm for 8 min, removing supernatant, and performing resuspension with sterile water; continuing to perform centrifugation at 25° C. and 3000 rpm for 3 min, and performing resuspension with sterile water; continuing to perform centrifugation at 25° C. and 6000 rpm for 2 min, removing supernatant, and performing resuspension with a TE-LiAC buffer solution; continuing to perform centrifugation at 25° C. and 6000 rpm for 2 min, removing supernatant, and performing resuspension with a TE-LiAC solution; sucking 100 μL of a resuspension solution, mixing the resuspension solution with 2 μL of a vector DNA (heated at 100° C. for 5 min, placed on ice, repeated twice) and 10 μL of an expression vector containing a yeast gene ScFIT3, mixing uniformly, and placing a mixture at 25° C. for 10 min; adding 260 μL of a PEG/TE-LiAC solution with a concentration of 40%, mixing uniformly, taking a water bath at 30° C. for 1 h, adding 43 μL of DMSO preheated at 37° C., mixing uniformly, performing heat shock at 42° C. for 5 min, performing centrifugation on a mixed solution subjected to heat shock at 12000 rpm for 30 s, and performing resuspension with sterile water; performing centrifugation on a resuspended solution at 12000 rpm for 30 s, performing resuspension with sterile water, coating 100 μL of the resuspended solution on an SC solid screening medium, and inverting and culturing same at 30° C. for 6 days to obtain the budding yeast JRY472 transformed with the yeast gene ScFIT3.

### Embodiment 3

1. The operation of this embodiment included: selecting the single colony of the budding yeast JRY472 (WT) transformed with the yeast gene ScFIT3 (ScFIT3) obtained in Embodiment 2, adding the single colony into 5 mL of the SC liquid screening medium, dispersing and mixing uniformly, and performing shake culture at 30° C. and 250 rpm for 10 h until OD600>0.3 is achieved to obtain a bacteria solution of the budding yeast JRY472 transformed with the yeast gene ScFIT3; diluting the bacteria solution of the budding yeast JRY472 transformed with the yeast gene ScFIT3 to OD600=0.1, continuing to perform shake culture at 30° C. and 250 rpm for 6 h, and then diluting the bacteria solution of the budding yeast JRY472 transformed with the yeast gene ScFIT3 to OD600=0.3, and taking the bacteria solution of the budding yeast JRY472 transformed with the yeast gene ScFIT3 obtained at this time as a mother solution; diluting the mother solution to a concentration gradient of 1/10, 1/100, and 1/1000 times, respectively; and culturing the bacteria solution of a wild-type budding yeast JRY472 to OD600=0.3, and diluting the bacteria solution at the same ratio.

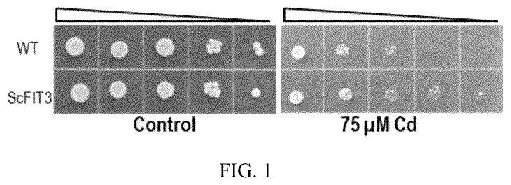

The operation further included: taking 3 μL of the mother solution and gradiently-diluted mother solutions, respectively, using the bacteria solution of the wild-type budding yeast JJRY472 and the gradiently-diluted solutions of the bacteria solution of the wild-type budding yeast JJRY472 as controls, dropping them onto an SC solid screening medium containing CdCl₂ (75 μM) and a normal SC solid screening medium, respectively, and inverting and culturing them at 30° C. for 4 days to obtain results as shown in FIG. 1.

It can be seen from FIG. 1 that the growth of the budding yeast transformed with the yeast gene ScFIT3 is significantly superior to that of a wild-type budding yeast, indicating that the budding yeast transformed with the yeast gene ScFIT3 has good resistance to cadmium stress, significantly enhances a capability of resisting heavy metal cadmium, and reduces accumulation and absorption of cadmium.
2. The operation included: inoculating the budding yeast JRY472 transformed with

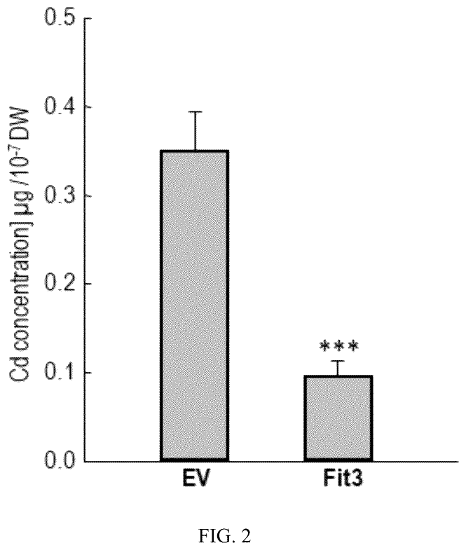

the yeast gene ScFIT3 and the wild-type budding yeast JRY472 in Embodiment 2 into a YPDA liquid medium, respectively; performing shake culture at 30° C. and 250 rpm until OD600=0.3 is achieved; and then, adding CdCl₂ to achieve a final concentration of 50 μM (an EV group and an FIT3 group, respectively); sampling after 14 h of adding CdCl₂, measuring the contents of Cd²⁺ in cells of the budding yeast JRY472 transformed with the yeast gene ScFIT3 and the wild-type budding yeast JRY472, respectively to obtain results as shown in FIG. 2.

It can be seen from FIG. 2 that the yeast of the wild-type budding yeast JRY472 has a cadmium content more than three times that of the budding yeast JRY472 transformed with the yeast gene ScFIT3, indicating that the budding yeast transformed with the yeast gene ScFIT3 has good resistance to cadmium stress, significantly enhances a capability of resisting heavy metal cadmium, and reduces accumulation and absorption of cadmium.

What is claimed is:

1. A yeast gene ScFIT3 for regulating and controlling cadmium resistance of an organism, wherein the yeast gene ScFIT3 for regulating and controlling the cadmium resistance of the organism has a nucleotide sequence as shown in SEQ ID NO.1.

2. An expression vector containing the yeast gene ScFIT3 for regulating and controlling cadmium resistance of a plant according to claim 1.

3. A method for constructing the expression vector according to claim 2, comprising the following steps:
(1) extracting an RNA from a budding yeast, and then performing reverse transcription on the RNA to obtain a cDNA;
(2) performing PCR amplification by using the cDNA as a template to obtain a sequence of a yeast gene ScFIT3, wherein PCR primer sequences are as follows:
Forward primer:
5′-ATGAAATTCTCTTCCGCTTT-3′,
Reverse primer:
5′-TTACAATAACATGACGGCAG-3′;
(3) linking the yeast gene ScFIT3 to a pRS416 plasmid to obtain the expression vector containing the yeast gene ScFIT3.

4. An application of the yeast gene ScFIT3 according to claim 1 in enhancing cadmium resistance of an organism.

5. The application according to claim 4, wherein a route of the application comprises:
transforming an expression vector containing the yeast gene ScFIT3 into a host organism.

6. The application according to claim 4, wherein the host organism comprises a plant and a microorganism.

7. The application according to claim 6, wherein the microorganism is a yeast.
